(19) United States
(12) Patent Application Publication
(10) Pub. No.: US 2018/0220620 A1
(43) Pub. Date: Aug. 9, 2018

(54) COMPOSITIONS AND METHODS FOR ODOR CONTROL

(71) Applicants: Dow Global Technologies LLC, Midland, MI (US); Rohm and Haas Company, Collegeville, PA (US)

(72) Inventors: Bruce D. Hook, Lake Jackson, TX (US); Kimberly A. Surber, Lake Jackson, TX (US); Karl V. Jacob, Midland, MI (US); Yi Fan, Midland, MI (US); Aslin Izmitli, Yardley, PA (US)

(73) Assignees: Dow Global Technologies LLC, Midland, MI (US); Rohm and Hass Company, Collegeville, PA (US)

(21) Appl. No.: 15/750,367

(22) PCT Filed: Aug. 4, 2016
(86) PCT No.: PCT/US2016/045585

§ 371 Date: Feb. 5, 2018

Related U.S. Application Data
(60) Provisional application No. 62/201,268, filed on Aug. 5, 2015.

(51) Int. Cl. A01K 1/015; A61L 9/012; A61L 9/04

(52) U.S. Cl. CPC A01K 1/0154; A61L 9/044; A61L 9/012; A01K 1/0155

(57) ABSTRACT

A composition comprising an inert material; a liquid binder; and an odor inhibitor, wherein the odor inhibitor is a salt of an aminopolycarboxylic acid compound. A method for preparing a composition comprising providing an odor inhibitor, wherein the odor inhibitor is a salt of an aminopolycarboxylic acid compound; providing an inert material, wherein the inert material comprises particles having a mean particle size less than 1000 microns; mixing the odor inhibitor the inert material in a high-shear mixer; introducing a liquid binder to the mixer; and mixing the liquid binder, the non-porous inert material and the odor inhibitor until granules form which are 200 to 2500 microns in diameter.

### FIELD

This invention relates generally to odor controlling compositions and methods.

### BACKGROUND

Animal litter is used as a catch material for feces and urine. These biological byproducts can develop strong odors due to evolution of malodorous compounds, especially ammonia Ammonia results from microbial action on urea and/or uric acid in the animal waste.

The odor from animal litters is distressing to humans and at sufficiently high levels, may be toxic. In addition, the odor is distressing to the welfare of the animals, especially in closed environments such as poultry houses and horse stalls/barns. Moreover, emissions from animal litter may contribute to the greenhouse effect. It is desirable, therefore, to find ways of controlling odors from animal litter.

Previous odor control compositions have had small particle size, as compared to the animal litter, which can result in the odor composition separating or segregating from the animal litter during packaging or transport to the consumer. Such segregation can cause the odor control composition to settle away from the surface of the animal litter, or otherwise become non-uniformly distributed in the animal litter, reducing the efficiency of the odor control composition. Likewise, proximity of the odor control components to the surface of the litter bed, or the outside of the granules is desirable.

The problem addressed by this invention is the provision of compositions and methods for preventing ammonia formation, and thereby controlling odor, in animal litters.

### STATEMENT OF INVENTION

In one instance, the present disclosure describes a composition comprising an inert material; a liquid binder; and an odor inhibitor, wherein the odor inhibitor is a salt of an aminopolycarboxylic acid compound.

In another instance, the present disclosure describes a method for preparing a composition comprising providing an odor inhibitor, wherein the odor inhibitor is a salt of an aminopolycarboxylic acid compound, wherein the odor inhibitor comprises particles; providing an inert material, wherein the inert material comprises particles having a mean particle size less than 1000 microns; mixing the odor inhibitor the inert material in a high-shear mixer; introducing a liquid binder to the mixer; and mixing the liquid binder, the non-porous inert material and the odor inhibitor until granules form which are 200 to 2500 microns in diameter.

### DETAILED DESCRIPTION

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

The term “prevent” as used herein means at least partly reducing the amount of ammonia that would otherwise be formed in animal waste in the absence of the additive. In some embodiments, the amount of ammonia is reduced by at least 50 percent, alternatively at least 70 percent, alternatively at least 90 percent, or alternatively 100 percent, as measured, for instance, by colorimetric indicators (e.g., using Drager tubes as described in the Examples), optical transmission absorption methods and/or gas chromatography in real time animal usage or laboratory testing method that mimics urine degradation.

The term “animal” as used in this specification generally means non-human animals. Non-limiting examples include domesticated animals, zoo animals, farm animals, pets, and other animals that spend some of their time in a partially or fully enclosed environment. More specific examples include, without limitation, cats, dogs, poultry (e.g., chickens), horses, cows, swine, rabbits, goats, and rodents (e.g., guinea pigs, hamsters, ferrets, mice, and rats).

The term “waste” refers to any animal waste product that may be transformed by bacteria into ammonia. In one instance, waste refers to any animal waste product that contains urea, uric acid, or both. Examples of waste include animal urine and excrement (e.g., feces, droppings).

The present disclosure describes a composition containing an inert material, a liquid binder and an odor inhibitor, and a method for preparing the composition. In one instance, the composition is combined with a carrier material. Unless specifically stated otherwise, when the present disclosure refers to the “composition” it does not refer to the combination of the composition and the carrier material. The carrier material may be any material that is typically used as a bedding or absorbent for animals and their waste and includes, for instance, swellable clays (e.g., bentonite and montmorillonite), non-swellable clays (e.g., kaolin), wood shavings, hay, wood chips, pelletized saw dust, paper, chopped corn cobs, peanut hulls, wood pulp, wheat grass, or mixtures thereof. In some embodiments, the carrier material is bentonite clay.

In some embodiments, the animal is a cat and the carrier material is bentonite clay. In some embodiments, the animal is a cat and the carrier material is wood chips, wood shavings, paper byproducts, pine pellets, ground corn cob or pelletized saw dust.

In some embodiments, the animal is poultry and the carrier material is wood shavings.

In some embodiments, the animal is a horse and the carrier material is wood chips, wood shavings, or pelletized saw dust.

The odor inhibitor used in the compositions and methods of the invention comprises a salt of an aminopolycarboxylic acid compound. As discussed above, the odor inhibitor functions by preventing formation of ammonia, an odor causing compound. Preferred cations for the salts of the invention include sodium or potassium.

In some embodiments, the aminopolycarboxylic acid salt has a pH in water from 4 to 9, preferably from 4 to 5.5.

In some embodiments, the odor inhibitor is an aminopolycarboxylic acid compound salt with an ethylenediamine or diethylenetriamine backbone.

In some embodiments, the odor inhibitor is an ethylenediaminetetraacetic salt, a diethylenetriaminepentaacetic salt, a N-hydroxyethylethylenediaminetriacetic salt, or a mixture of two or more thereof.

In some embodiments, the odor inhibitor is a sodium salt of ethylenediaminetetraacetic acid, a potassium salt of ethylenediaminetetraacetic acid, or a mixture thereof.

In some embodiments, the odor inhibitor is a sodium salt of ethylenediaminetetraacetic or a mixture of such salts. For instance, the odor inhibitor is monosodium ethylenediaminetetraacetic acid (NaEDTA), disodium ethylenediaminetetraacetic acid (Na₂EDTA), trisodium ethylenediaminetetraacetic acid (Na₃EDTA), tetrasodium ethylenediaminetetraacetic acid (Na₄EDTA), or a mixture of two or more thereof. In some embodiments, Na₂EDTA is preferred.

In some embodiments, the odor inhibitor is a potassium salt of ethylenediaminetetraacetic acid or a mixture of such salts. For instance, the odor inhibitor is monopotassium ethylenediaminetetraacetic acid (KEDTA), dipotassium ethylenediaminetetraacetic acid (K₂EDTA), tripotassium ethylenediaminetetraacetic acid (K₃EDTA), tetrapotassium ethylenediaminetetraacetic acid (K₄EDTA), or a mixture of two or more thereof. In some embodiments, K₂EDTA is preferred.

In some embodiments the inert material is a material in the nature of particulates or granules which may be combined with the odor inhibitor as described herein to form the composition. In one instance, the inert material is a non-porous inert material. Examples of non-porous inert materials include citrate salts, talc, gypsum, calcium carbonate, sand, glass, and dirt. In one instance, the inert material is a porous inert material. Examples of porous inert materials include diatomite, alumina, alumina silicates and silicates. In one instance, it is preferred that the inert material is a non-porous inert material. In one instance, it is preferred that the inert material is substantially calcium-free, for example, the inert material contains less than 1 percent calcium by weight. Without being limited by theory, it is assumed that the odor inhibitor chelates calcium-containing materials. In some instances, the preferred inert material is sand. In some instance, the preferred inert material is a mixture of sand and a citrate salt. In some instances, the inert material is substantially pore-free, meaning the material is comprised of fewer than 90% voids as calculated by total volume. Preferably, the inert material has a neutral pH. In one instance, the inert material has a pH of 6 to 8, in one instance the pH is 6.5 to 7.5, in one instance the pH is 6.8 to 7.2, in one instance the pH is 6.9 to 7.1, in one instance the pH is 7.0. Preferably, the inert material has low toxicity. As used herein, “low toxicity” refers to a compound that has a lethal dosage (as measured by the oral LD50 standard) of greater than 1000 milligrams of compound per kilogram bodyweight.

In some embodiments, the liquid binder is a liquid solution which, when combined with the odor inhibitor and the inert material, combines the odor inhibitor and inert material into granules such that during transport and typical use, a majority of the granules will substantially retain their shape and size. In some instances the liquid binder is water, salt solutions, solutions of EDTA salts, glycols, propylene glycol, glycerin, polyethylene glycol, polypropylene glycol, polyalkylene glycol lubricants, or a combination thereof. Preferably, the liquid binder is a liquid at room temperature. Preferably, the liquid binder has low toxicity. Some of the listed liquid binders will be subject to some evaporation at room temperature, as such, once the granules are formed it is anticipated, depending on the liquid binder selected, at least a portion of the liquid binder will evaporate out of the granules thereby lowering the liquid binder content in the granules. It is observed that the granules hold together even after a portion of the liquid binder evaporates. Without being limited by theory, it is expected that as the granules are initially held together by the capillary action of the liquid binder, but as the odor inhibitor and inert material contact each other the crystal structure of either or both of the odor inhibitor and inert material is modified to bind the odor control inhibitor to the inert material such that the granules remain intact following at least partial evaporation of the liquid binder.

In another aspect, the invention provides improved efficiency in prevention of urea degradation due to the preferred placement of the odor control agent at or near the surface of the litter particles.

The compositions and methods described herein may contain other additives, besides the odor inhibitor and carrier material, that are typically used in animal litters. These include, but are not limited to, fillers, humectants, disintegrants, odor absorbing materials (e.g., sodium carbonate, potassium carbonate, siliceous material, opaline silica, activated carbon, sodium bisulfate complex, or corn starch), zeolite, dedusting agents (e.g., gaur gum, PTFE coated clay, or fluoropolymers), antimicrobials such as bronopol and silver based compounds, fragrances, other chelants (diethylenetriaminepentaacetic acid (DTPA) for example), gypsum, small molecule organic acids, polymers with neutralization capacity or acid groups (e.g., cellulose acetate, polyxcarboxylates), rice flour, quaternary amines, probiotic bacteria and/or ammonia oxidizing bacteria.

In some embodiments, the composition described herein is free of other odor preventing additives, for instance it is free of one or more of: an alkali metal tetraborate n-hydrate, alum, other transition metal salts (e.g., Zn, copper salts) and/or boron compounds. When it is said that the composition is free of an additive, it means that the composition contains less than 1 weight percent of that additive, preferably less than 0.1 weight percent of that additive, more preferably, less than 0.01 weight percent of that additive.

In some embodiments, the granules of the composition have a mean particle size greater than 100 microns and less than 2500 microns. In some embodiments, the composition has a mean particle size greater than 200 microns and less than 2500 microns. In some embodiments, the composition has a mean particle size greater than 200 microns and less than 1500 microns.

In some embodiments, the composition comprises 10 to 95 percent by weight inert material. In some embodiments, the composition comprises 15 to 80 percent by weight inert material. In some embodiments, the composition comprises 20 to 70 percent by weight inert material. In some embodiments, the composition comprises 25 to 60 percent by weight inert material. In some embodiments, the composition comprises 30 to 50 percent by weight inert material. In some embodiments, the composition comprises 35 to 45 percent by weight inert material.

In some embodiments, the composition comprises 5 to 90 percent by weight odor inhibitor. In some embodiments, the composition comprises 10 to 85 percent by weight odor inhibitor. In some embodiments, the composition comprises 15 to 80 percent by weight odor inhibitor. In some embodiments, the composition comprises 20 to 75 percent by weight odor inhibitor. In some embodiments, the composition comprises 25 to 70 percent by weight odor inhibitor. In some embodiments, the composition comprises 30 to 65 percent by weight odor inhibitor. In some embodiments, the composition comprises 35 to 60 percent by weight odor inhibitor. In some embodiments, the composition comprises 40 to 55 percent by weight odor inhibitor. In some embodiments, the composition comprises 44 to 54 percent by weight odor inhibitor. In some embodiments, the composition comprises 46 to 53 percent by weight odor inhibitor. In some embodiments, the composition comprises 48 to 52 percent by weight odor inhibitor. In some embodiments, the composition comprises 49 to 51 percent by weight odor inhibitor.

In some embodiments, the composition comprises 0.01 to 15 percent by weight liquid binder. In some embodiments, the composition comprises 0.1 to 14 percent by weight liquid binder. In some embodiments, the composition comprises 1 to 13 percent by weight liquid binder. In some embodiments, the composition comprises 4 to 12 percent by weight liquid binder. In some embodiments, the composition comprises 8 to 11 percent by weight liquid binder.

In some embodiments, the dispersion rate of the composition is rapid, such that when the composition is contacted by the waste, the odor inhibitor and binder will rapidly dissolve and combine with the waste to allow the odor inhibitor to act to prevent odor formation. Rapid dispersion allows the composition to prevent the formation of ammonia before the bacteria in the waste is able to produce a substantial amount of ammonia. As shown in the Examples herein, the dispersion rate of the composition described herein is faster than previous odor inhibiting compositions, and as such the present composition is an improvement.

In some embodiments, the composition described herein is formed according to the following method: (a) providing an odor inhibitor, wherein the odor inhibitor is a salt of an aminopolycarboxylic acid compound; (b) providing an inert material, wherein the inert material comprises particles having a mean particle size less than 1000 microns; (c) mixing the odor inhibitor the inert material in mixer; (d) introducing a liquid binder to the mixer; (e) mixing the liquid binder, the non-porous inert material and the odor inhibitor until granules form which are 200 to 2500 microns in diameter. In some embodiments, the granules are dried. In one instance, the drying step is performed prior to a screening step. In some embodiments, the composition is further formed by screening the composition to remove particles less than 200 microns in diameter and particles greater than 2500 microns in diameter. When referring to particle size, it is understood that the granules and particles describes herein will be irregular shape, and as such when a particle is described as having a particle size, for example, 200 microns in diameter, it is understood that this means that the average size of the particles is 200 microns. In a preferred embodiment, the composition is sized such that the granules are not an inhalation risk, such as can be the case with smaller particles, especially particles smaller than 150 microns.

The drying step may be done by heat or some other method, for example, convection. In one instance, the drying step results in at least a portion of the liquid binder evaporating from the composition. In one instance, the drying step removes at least 10 percent by weight of the liquid binder from the composition. In one instance, the drying step removes at least 20 percent by weight of the liquid binder from the composition. In one instance, the drying step removes at least 30 percent by weight of the liquid binder from the composition. In one instance, the drying step removes at least 40 percent by weight of the liquid binder from the composition. In one instance, the drying step removes at least 50 percent by weight of the liquid binder from the composition. In one instance, the drying step removes at least 60 percent by weight of the liquid binder from the composition. In one instance, the drying step removes at least 70 percent by weight of the liquid binder from the composition. In one instance, the drying step removes at least 80 percent by weight of the liquid binder from the composition. In one instance, the drying step removes at least 90 percent by weight of the liquid binder from the composition.

The mixer used in the method described herein is any mixer sufficient to form the granules described, for example, a high-shear mixer.

In one instance, the odor inhibitor is defined by particles where 90 percent of the particles are less than 100 microns and 50 percent of the particles are less than 25 microns.

The addition of the composition to the carrier material, as described herein, results in the prevention of ammonia, thereby significantly reducing undesirable animal odors. The composition can be incorporated with the carrier material by a variety of standard techniques known to those skilled in the art including, for instance, solids mixing (including dry blending or co-grinding), spreading, sprinkling, and the like.

For instance, in one embodiment, the composition may be sprinkled over the top of a bed of the carrier material. In this embodiment the bed of carrier material may be further agitated to mix the composition deeper into the material.

In another embodiment, the composition may be dry blended with the carrier material and packaged together prior to use as an animal litter. In a preferred embodiment, the composition is made into granules prior to dry blending such that the granules and the carrier material are of a similar size and shape so to inhibit demixing or stratification of the odor inhibitor containing particles from the carrier due to segregation.

In any of the embodiments above, different equipment may be used in order to accomplish the incorporation of the composition and the carrier material.

When mixing the composition, it may be desirable to match the particle density as well as shape of the carrier and composition to reduce the likelihood of particle segregation.

Other mixing techniques may include dry briquetting the mixture of carrier material and composition to have both incorporated uniformly in the process (e.g., a size range from hundreds of microns to millimeters may be suitable).

In some embodiments, the composition may be applied to the carrier material prior to the animal releasing its waste on the carrier material. In some embodiments, the composition may be applied or reapplied to the carrier material for second or subsequent generations of use.

A person of ordinary skill in the art can readily determine the effective amount of odor inhibitor compounds of the invention that should be included in the composition, which composition is used in combination with the carrier material. By way of non-limiting example, suitable amounts of the odor inhibitor may include at least 0.01 weight percent, preferably at least 0.1 weight percent, more preferably at least 0.15 weight percent, based on the total weight of the combination of the carrier material and the composition. Although there is no particular upper limit on the amount of the odor inhibitor, in some embodiments it may be desirable to use 10 weight percent or less, alternatively 8 weight percent or less, alternatively 5 weight percent or less, alternatively 1.2 weight percent or less, or alternatively 0.5 weight percent or less, based on the total weight of the combination of the carrier material and the composition.

The addition of the odor inhibitor to an animal litter containing a carrier material reduces undesirable animal odors. The odor inhibitor can be incorporated with the carrier material by a variety of standard techniques known to those skilled in the art including, for instance, spraying, solids mixing (including dry blending or co-grinding), spreading, sprinkling, crystallization/precipitation of the odor inhibitor onto the carrier material, and the like.

For instance, in one embodiment, the odor inhibitor may be sprinkled over the top of a bed of the carrier material. In this embodiment the bed of carrier material may be further agitated to mix the odor inhibitor deeper into the material. The sprinkled odor inhibitor may also include a carrier or a flow aid that helps disperse the odor inhibitor throughout the litter bed.

In another embodiment, the odor inhibitor may be dry blended with the carrier material and packaged together prior to use as an animal litter. In a preferred embodiment, the odor inhibitor is made into a fine powder prior to dry blending to enhance the coatability of the odor inhibitor onto the carrier material. The fine powder may be produced by spray drying, grinding, precipitation or combination thereof, and may be combined with a flow aid by dry blending or binding with a liquid to enhance attachment to the carrier. In another preferred embodiment, a liquid is mixed in with the odor inhibitor powder and the carrier to aid with binding the odor inhibitor onto the carrier. That liquid may, for instance, be water, or a solvent or an oil or a solution of the odor inhibitor, or any liquid whose presence enhances the adhesion of the odor inhibitor powder to the carrier.

In another embodiment, the carrier material may be sprayed with a binder liquid and then blended with dry odor inhibitor so that the dry particles are attached to the carrier material by the binder liquid at a desired concentration. Likewise, the carrier material may be blended with dry odor inhibitor and then sprayed with a binder liquid so that the dry particles are attached to the carrier material by the binder liquid at a desired concentration. The coated carrier particles may then optionally be dried in order to evaporate water or a solvent component that may optionally be present in the binder liquid. Preferably, the binder is at least partially water soluble. Possible binders may comprise solutions of starch, water, polyethylene glycol, polypropylene glycol, or the odor inhibitor itself.

Some embodiments of the invention will now be described in detail in the following Examples.

### EXAMPLES

Synthetic Urine Preparation.

In these examples, synthetic urine is prepared as follows. In a sterilized bottle add the following components: Urea (USP Grade): 85.0 g, Sodium Chloride (Morton's® Table Salt or USP Grade): 9.0 g, Magnesium Sulphate Pentahydrate (ACS Grade): 0.4 g, Calcium Acetate (99% purity): 0.7 g Potassium Sulfate (ACS Certified): 4.0 g, and Ammonium Sulfate (ACS Certified): 2.4 g. 1000 g of reverse osmosis purified water is added to a separate sterilized bottle. The purified water is poured into the bottle containing the components. The bottle is capped and placed on a bottle roller at ambient temperature, for example for 1 to 3 hours. The pH of the solution is measured to confirm the pH is between 6.5 and 7. If the pH is greater than 7 then ammonium sulfate is added to bring the pH into range (generally 0.2 to 0.3 g).

Bacteria Preparation.

In these examples, the bacteria used for the efficacy testing is an environmental isolate from a cat's feces. This pure strain is isolated on a sterile tryptic soy agar plate and stored in the refrigerator. A fresh plate is prepared every 2 weeks. This strain is sent to an external lab for DNA sequencing and determined that it is a strain of staphyloccocus xylosus. The typing strain of staphylococcus xylosus from ATCC with the catalog number 29971 is tested and gave similar results to the environmental isolate used here at higher concentrations.

The bacteria is grown the day before the start of the experiments by taking a loopful of the bacteria from the agar plate with a 10 uL loop and immersing it in 10 mL sterile tryptic soy broth filling half of the container. This is an aerobic bacteria, therefore air is needed on top of the growth medium. The vial is incubated at 30 degrees C. in a shaking incubator at 100 rpm for 24 hours. The count of the bacteria in the growth media can be determined by the serial dilutions method, where 10 serial dilutions of the growth medium in sterile Physiological Buffered Saline (PBS) solution are performed with 10× dilution each time. Each dilution is plated twice using 100 uL of growth media for each plate. The number of colonies are counted on the dilution plate that has between 30 and 300 colony forming units (CFU) on it. The number of colony forming units in the original growth media is then determined by back-calculating the number of dilutions.

Typically the bacterium used for here grows to ˜9.9*10⁷ CFU/mL overnight (this estimate is used for doing the dilutions before inoculating samples).

All pipettes, containers, tips, spoons etc. equipment used for these experiments are sterile in order to prevent contamination with other bacteria. Litters and standalone treatments are used as-is, without sterilization.

### Example 1

Composition Preparation:

To a food processor mixer is added 50.0 grams K₂EDTA, 50.0 grams 40-70 mesh sand and 5 milliliters propylene glycol, and then are mixed for 120 seconds. The resulting composition is screened to separate the composition by granule size: 10-18 mesh, 18-40 mesh and greater than 40 mesh (pan).

Litter Preparation.

Charge a sterilized 1 L (ex. Tri-Pour®) container with 190 g of cat litter to be tested (National 12 bentonite clay litter from Bentonite Performance Materials LLC). Weigh and set aside 10 g of litter for each test sample. Level the litter in each test sample by tapping on the container. Create a depression approximately 17 mm in depth and 27.5 mm in diameter in the center of the litter sample, using a clean 50 mL conical tube. Wipe the outside of the tube in between test samples. 6.1 grams of granules of the composition is sprinkled in the depression formed in each size range is sprinkled on top of 200 grams of litter (National 12 bentonite clay), only a single granule size range being added to each depression. Prepare the inoculation solution by diluting the bacteria solution in tryptic soy broth with sterile PBS to get 5*10⁷ CFU/mL Immediately before inoculation of each sample, add 3 mL of the inoculation solution into 37 mL of synthetic urine in a sterile 50 mL centrifuge container, vortex mix it for 3 seconds and pour it in the depression in the litter sample. 40 milliliters of synthetic urine is added to a bacterial inoculum with approximately 3×10⁻⁷ cells per milliliter, and the combination thereof is added to the top of the treated litter. Evenly sprinkle the 10 g of litter which was set aside on the top of the synthetic urine mixture. An aluminum foil sheet is used to cover the beaker to prevent vapors from escaping the beaker.

Headspace Ammonia Measurement.

Draeger tubes are used to sample the headspace of each test container is sampled using a SENSIDYNE® AP-20S Aspirating Detector Tube Pump. Low (0.2-20 ppm) or high (5-260 ppm) range SENSIDYNE® Ammonia Gas Detector Tubes are used to quantify the headspace ammonia concentration. The following procedure is used for ammonia detection and quantification in each test sample. 1. Break both ends of the detector tube using the breaking port on the pump. 2. Point the arrow mark on the detector tube towards the aspirating pump. Insert the detector tube securely into the rubber tube connector of the aspirating pump. 3. To sample the test headspace, pierce a small hole in the center of the aluminum foil cover of the test sample. Insert the detector tube (attached to the aspirating pump) into the sampling hole to the specified measurement distance. Insert the tube into the headspace to 45 mm, equal to the thick blue line on the bottom end of the gas detector tube). 4. Hold the pump at the 45 mm measurement distance. Pull the pump handle at full stroke to the locked position. Wait for 1 minute until sampling is complete which is confirmed with the flow indicator of the pump. The instruction manual of the aspirating pump will give more details if necessary. Read the scale at the maximum point of the stained layer (yellow in color). Read and report the concentration immediately after measurement. If the reading is off scale, for example 20 ppm for the low range tubes, repeat the measurement using the high range gas detector tube. After sampling, seal the sampling hole in the aluminum foil cover with tape. Sampling will be conducted on multiple days throughout the test, as specified by the testing conditions. The sampling is done on days 1, 3, 6, and 10 or 1, 4, 7, 11. measure the ammonia levels generated. The results are summarized in Tables 1 and 2.

TABLE 1

| | Actual | | | | | |
|---|---|---|---|---|---|---|
| | bacterial | | | Particle | Standalone | % active, |
| | inoculation | Treatment | Standalone | Size | amount | M2EDTA in |
| Treatment | (CFU/mL) | (ppm) | Sample | (Mesh) | (gm) | standalone |
| A | 2.85E+07 | 16700 | 60D | 10-18 | 6.1 | 56.4 |
| B | 2.85E+07 | 12400 | 60D | 18-40 | 6.1 | 41.8 |
| C | 2.85E+07 | 12200 | 60D | pan | 6.1 | 41.1 |

TABLE 2

| | | Avg. | | Avg. |
|---|---|---|---|---|
| | Avg. Ammonia | Ammonia | Avg. Ammonia | Ammonia |
| Treatment | (ppm) Day 1 | (ppm) Day 3 | (ppm) Day 6 | (ppm) Day 9 |
| A | 0.0 | 0.0 | 0.0 | 0.1 |
| B | 0.1 | 0.3 | 0.8 | 1.4 |
| C | 0.4 | 1.6 | 2.6 | 3.8 |

The results indicate that there is a reduction in ammonia generation by mixing the odor control formulation with cat litter.

### Example 2

Composition Preparation:

To a food processor mixer is added 50.0 grams Na₂EDTA, 50.0 grams 40-70 mesh sand and 5 milliliters of a solution containing 40% K₂EDTA in water, and then are mixed for 120 seconds. The resulting composition is screened to separate the composition by granule size: 10-18 mesh, 18-40 mesh and greater than 40 mesh (pan).

Litter Preparation.

Litter is prepared according to Example 1.

Headspace Ammonia Measurement Ammonia is measured as described in Example 1.

The results are summarized in Tables 3 and 4.

TABLE 3

| | Actual | | | Particle | Standalone |
|---|---|---|---|---|---|
| Treat- | inoculation | Treatment | Standalone | Size | amount |
| ment | (CFU/mL) | (ppm) | Sample | (Mesh) | (gm) |
| A | 4.55E+07 | 5000 | 72B | 18-40 | 2.2 |
| B | 4.55E+07 | 15000 | 72B | 18-40 | 6.5 |
| C | 4.55E+07 | 15000 | 72B | 10-18 | 6.5 |
| D | 4.55E+07 | 15000 | 72B | pan | 6.5 |

TABLE 4

| | Avg. Ammonia | Avg. Ammonia | Avg. Ammonia | % active, |
|---|---|---|---|---|
| Treatment | (ppm) Day 1 | (ppm) Day 3 | (ppm) Day 6 | M2EDTA |
| A | 0.6 | 2.2 | 4.0 | 40.6 |
| B | 0.0 | 0.3 | 1.0 | 40.6 |
| C | 0.0 | 0.5 | 1.0 | 64.1 |
| D | 0.5 | 0.9 | 1.5 | 44.6 |

### Example 3

Composition Preparation:

To a 10 liter double arm sigma mixer (LCI Corp. model KDHJ-10) is added 1200 gm of sand (40-70 mesh), 300 gm sodium citrate hydrate powder, and 1500 gm Na2EDTA powder. The dry powders are blended for 120 seconds in the mixer. 150 ml of deionized water is added slowly over 15 minutes while mixing. The wetted powders are then blended for an additional 5 minutes. The granulated powder blend is then removed to a container. From the container the blend is then spread onto three baking trays and placed into an oven for 30 minutes (oven temperature set point is 105° C.). After 30 minutes, the dried granules are removed from the oven and screened. Oversize clumps are broken up and rescreened. From the screening the following products were obtained: 1805 gm of granules between 360 and 2450 microns, 787 gm of powder smaller than 360 microns and 8 gm particles larger than 2450 microns.

The granules sized between 360 and 2450 microns are tested for efficacy with litter in the same manner as Example 1.

Litter Preparation.

Litter is prepared according to Example 1.

Headspace Ammonia Measurement.

Ammonia is measured as described in Example 1. The results are summarized in Tables 5 and 6.

TABLE 5

| | Actual | | | Particle | Standalone |
|---|---|---|---|---|---|
| Treat- | inoculation | Treatment | Standalone | Size | amount |
| ment | (CFU/mL) | (ppm) | Sample | (Mesh) | (gm) |
| A | 5E+07 | 9600 | M150714A | 8-40 | 6.5 |

TABLE 6

| | Avg. Ammonia | Avg. Ammonia | Avg. Ammonia | % active, |
|---|---|---|---|---|
| Treatment | (ppm) Day 1 | (ppm) Day 6 | (ppm) Day 10 | M2EDTA |
| A | 0.4 | 3.1 | 5.2 | 30.5 |

1-8. (canceled)

9. A method for preparing a composition comprising:
(a) providing an odor inhibitor, wherein the odor inhibitor is a salt of an aminopolycarboxylic acid compound, wherein the odor inhibitor comprises particles;
(b) providing an inert material, wherein the inert material comprises particles having a mean particle size less than 1000 microns;
(c) mixing the odor inhibitor the inert material in a mixer;
(d) introducing a liquid binder to the mixer; and
(e) mixing the liquid binder, the non-porous inert material and the odor inhibitor until granules form which are 200 to 2500 microns in diameter.

10. The method of claim 9, further comprising drying the composition.

11. The method of claim 9, wherein the inert material comprises diatomite, citrate salts, talc, gypsum, calcium carbonate, sand, glass, dirt, alumina, alumina-silicates, silicates, or a combination thereof.

12. The method of claim 9, wherein the liquid binder comprises water, salt solutions, solutions of EDTA salts, glycols, propylene glycol, glycerin, polyethylene glycol, polypropylene glycol, UCON lubricants (food grade)

13. The method of claim 9, wherein the odor inhibitor is a salt of an aminopolycarboxylic acid compound having an ethylenediamine or diethylenetriamine backbone.

14. The method of claim 9, wherein the odor inhibitor is a salt of ethylenediaminetetraacetic acid, a salt of diethylenetriaminepentaacetic acid, a salt of N-hydroxyethylethylenediaminetriacetic acid, or a mixture of two or more thereof.

15. The method of claim 9, wherein the odor inhibitor is the disodium salt of ethylenediaminetetraacetic acid, the dipotassium salt of ethylenediaminetetraacetic acid, or a mixture thereof.
